(19) United States
(12) Patent Application Publication
(10) Pub. No.: US 2026/0166594 A1
(43) Pub. Date: Jun. 18, 2026

(54) MENSTRUAL CUP CLEANING AND STERILISING CASE

(71) Applicant: KGMW LTD., Greater London (GB)

(72) Inventors: Kira Goode, Greater London (GB); Monica Wai, Greater London (GB)

(21) Appl. No.: 19/129,487

(22) PCT Filed: Nov. 8, 2023
(86) PCT No.: PCT/GB2023/052910

§ 371 Date: May 13, 2025

(30) Foreign Application Priority Data
Nov. 16, 2022 (GB) 2217149.0

(51) Int. Cl. B08B 3/04; A61F 5/44; A61F 5/455; A61L 2/04; A61L 2/18; A61L 103/45; B08B 3/10

(52) U.S. Cl. CPC B08B 3/044; A61F 5/4404; A61F 5/4553; A61L 2/04; A61L 2/18; B08B 3/10; A61L 2103/45; B08B 2203/007

(57) ABSTRACT

A container for a menstrual cup, comprising a tubular body including a first end with an opening to admit the wide end of the cup and a second end which tapers inwardly to define an inner surface for supporting the wide end of the cup. The inner surface has formations providing a fluid communication path between the inside of the body outside the cup and a space in front of the wide end of the cup. A first end cap is removably attached to the first end of the body such that the first end cap, tubular body, and tapering inner surface define a space for the cup. A second end cap is removably attached over the second end of the body such that the tubular body with the first and second end caps defines a container for the cup. The body can be filled with a cleaning fluid, a cup inserted, and the body shaken to clean the cup. The cup can be sterilised by placing a cup in the body filled with a cleaning fluid and heated.

### FIELD

This invention relates to a container device or case that can be used for cleaning and sterilising a menstrual cup.

### BACKGROUND

A menstrual cup is a menstrual hygiene device which is inserted into the vagina during menstruation. Its purpose is to collect menstrual fluid (blood from the uterine lining mixed with other fluids). Menstrual cups are usually made of flexible materials such as medical grade silicone, latex, or a thermoplastic isomer. One example is a bell-shaped cup as shown in FIG. 8, open at a wide end 13 and with a stem 14 or a ring at a narrow, closed end. The stem 14 is used for insertion and removal, and the bell-shaped cup seals against the vaginal wall just below the cervix and collects menstrual fluid.

When a user of a menstrual cup wants to clean their menstrual cup after use, they need to rinse their cup out with clean water. When they want to sterilise their menstrual cup they can, for example, boil it either in a pan full of clean water on the hob for 15 minutes, or in a container full of clean water in a microwave for 3 minutes.

The current recommended process for cleaning a menstrual cup is to rinse it out in a sink. However, this is not a practical solution in a public toilet or washroom setting. Often the sinks are shared with other members of the public and washing menstrual blood in the presence of other people is often regarded as unacceptable practice. Furthermore, in the time it takes to leave a stall, rinse and clean the cup in the sink, and re-enter the stall to re-insert the cup, the user may experience period leakages.

Typically, a user will use a cooking pan or a mug to conduct the sterilisation process. This then leads to that user needing to have a spare pan dedicated to sterilisation because of the concerns around the cross-contamination of utensils that are traditionally used for cooking, eating, or drinking with menstrual blood.

BE1028521 discloses a device for cleaning and sterilizing a menstrual cup consisting of a base and a container made of detachable parts comprising a body with an internal cavity and an upper opening and a lower opening, for receiving a menstrual cup having a stem, a detachable cover, called the upper cover, for closing the upper opening of the body, a cover, called the lower cover, for closing the lower opening, portions in the internal cavity for holding and supporting the menstrual cup inside the device, and suitable to receive a cleaning liquid; the different parts being made of a rigid biocompatible material; in which the container is complementary to the base, meaning that it can be attached to the base, thus creating an electronic or thermal connection between the two, so that the dimensions of the body of the container are adapted to the average size of a menstrual cup. A method is also disclosed for optimizing the use of menstrual cups comprising subsequent cleaning and sterilization steps of the menstrual cup, characterized in that the cleaning and sterilization of the menstrual cup can be carried out in the same object.

This invention aims to provide a container device that is convenient for cleaning and sterilising a menstrual cup.

### Statement of Invention

A first aspect of the invention provides a container for a menstrual cup comprising an open end and a closed end, the container comprising:
a tubular body including:
a first end with an opening that is wide enough to admit the open end of a menstrual cup; and
a second end at which the tubular body tapers inwardly to define an inner surface for supporting the open end of a menstrual cup inserted into the body, wherein formations are defined in the inner surface such that when the open end of a menstrual cup is supported on the inner surface, a fluid communication path is provided between the inside of the tubular body around the outside of the menstrual cup and a space in front of the open end of the menstrual cup;
a first end cap that is removably attached to the first end of the tubular body in a fluid-tight manner such that when the first end cap is attached to the tubular body, the first end cap, tubular body, and tapering inner surface define a space within which a menstrual cup can be contained; and
a second end cap that is removably attached over the second end of the tubular body in a fluid-tight manner;
such that the tubular body with the first and second end caps attached in a fluid-tight manner define a fluid-tight container for a menstrual cup.

The second end of the tubular body can include an opening that is narrower than the opening at the first end and is not wide enough to admit the open end of a menstrual cup. The second end cap can include a seal that seals around the opening in the second end of the tubular body in a fluid-tight manner when the second end cap is attached to the second end of the tubular body.

The first and second end caps can be substantially the same and each include an outer seal for sealing around an outer part of the tubular body, and an inner seal for sealing around the opening in the second end. The first and second end caps can each be flat in a central area. This allows the container to stand upright on a flat surface. By providing end caps that are substantially the same, errors in reassembling the container by attaching the wrong end cap to the wrong end can be avoided.

The formations in the inner surface can be raised portions or recessed portions spaced around the inner surface.

The tubular body can include an indicator on its outer surface showing which end is the first end and which end is the second end.

An elastomeric sleeve can be provided around the tubular body. The elastomeric sleeve and the tubular body can have inter-engaging notches. In one embodiment, the tubular body has a first ridge around its outer surface with a notch formed in the first ridge; and the elastomeric sleeve has a corresponding notch-shaped cutout in its edge at one end. The first ridge can be adjacent the first end of the tubular body. The tubular body can also have a second ridge around its outer surface spaced from the first ridge and adjacent the second end of the tubular body, and the elastomeric sleeve can be located between the first and second ridges. The notch on the tubular body has a wide opening and a point, and wherein the wide part is closer to the first end of the tubular body and the point is closer to the second end of the tubular body. The notch can function as the indicator showing which end is the first end and which end is the second end A second aspect of the invention provides a method of using the container, comprising:
filling the container with a cleaning liquid;
removing the first end cap;
inserting a menstrual cup through the opening in the first end so that the open end is supported on the inner surface at the second end;
attaching the first end cap to the tubular body;
shaking the container to clean the menstrual cup with the cleaning liquid;
opening the first end cap;
removing the menstrual cup; and
reattaching the first end cap.

A third aspect of the invention provides a further method of using the container, comprising:
removing the first end cap;
inserting a menstrual cup through the opening in the first end so that the open end is supported on the inner surface at the second end;
filling the container with a cleaning liquid;
heating the container and menstrual cup to a sterilising temperature for a period of time;
removing the menstrual cup; and
draining the cleaning liquid from the container.

Further aspects of the invention are described below in relation to the drawings.

### DESCRIPTION OF THE DRAWINGS

The invention will now be described solely by way of example and with reference to the accompanying drawings in which:

FIG. 1 shows the assembly of all the parts that make up a menstrual cup cleaning and sterilising case according to an embodiment of the invention.

FIG. 2 shows the fully assembled menstrual cup cleaning and sterilising case of FIG. 1.

FIG. 3 shows a side view of the tubular main body of the menstrual cup cleaning and sterilising case of FIG. 1.

FIG. 4 shows an axial inside view of second end of the tubular main body of the menstrual cup cleaning and sterilising case of FIG. 1.

FIG. 5 shows a side view of the end cap or lid of the menstrual cup cleaning and sterilising case of FIG. 1.

FIG. 6 shows a perspective view of the inside of the end cap or lid of FIG. 5.

FIG. 7 shows a cross-sectional view of a fully assembled menstrual cup cleaning and sterilising case of FIG. 1 with a menstrual cup in it.

FIG. 8 shows an example of a menstrual cup.

### DETAILED DESCRIPTION

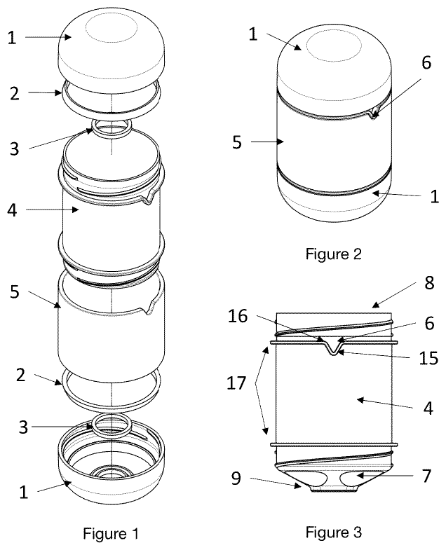

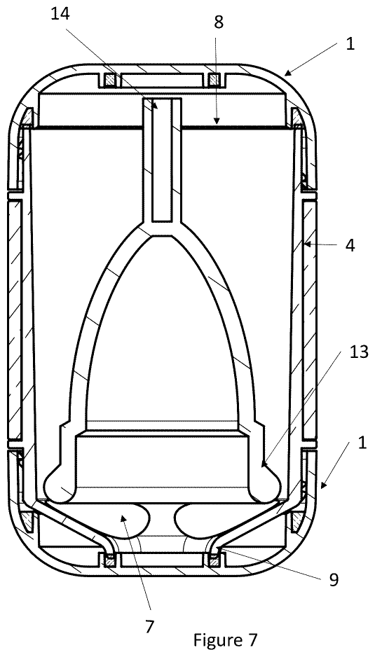

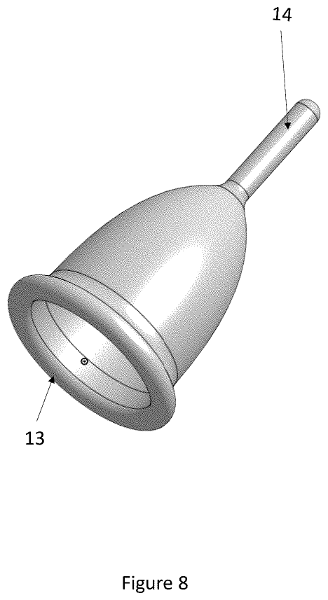

As shown in FIGS. 1, 2, 3, and 7 the case or container comprises a tubular main body 4 with openings 8, 9 at either end. The opening 8 at the first end is large enough so that the (open) wide end 13 of a menstrual cup (FIG. 8) can be inserted through it. The tubular body 4 tapers at its second end to an opening 9 that is small enough that the wide end 13 of menstrual cup cannot fit through.

The outer surface of the main body 4 has ridges around either end separate by a gap 17 with a notch 6 in the upper ridge to provide a visual and tactile indication to the user which opening is the wide opening end 8 and which opening is the tapering funnel end 9 (other visual and tactile indicators can be used). The point 15 of notch 6 is closer to the funnel end 9 of the main body 4 and the opening 16 of notch 6 is closer to the opening end 8 of the main body 4.

FIG. 2 shows a notched sleeve 5 pulled over the main body 4 and lined up so as to align with the notch 6. The sleeve 5 is made from an elastomeric flexible material so that it can stretched over the main body 4 in the gap 17 so that as a fully assembled unit, the sides are completely smooth to the touch. The sleeve 5 is made from a material that can add grip to the product when being used—so it doesn't slip out easily from the user's hand when they shake it. The ridges and notches assist in holding the sleeve in position.

A lid 1 is screwed onto the end of the main body 4 at either end to provide a fluid-tight seal around the openings 8, 9. As shown in FIG. 6, a smaller seal ring 3 is fixed inside lid 1 within the gap or groove 11, and larger seal ring 2 is fixed inside lid 1 in the gap or groove 12. Both lids 1 have a seal 2 for the opening end 8 and a seal 3 for the funnel ending 9 and are identical so that they are interchangeable on the main body 4. One lid 1 is screwed onto the opening end 8 of the main body 4, and the second lid 1 is screwed onto the funnel end 9 of the main body 4. This makes up the full assembled product.

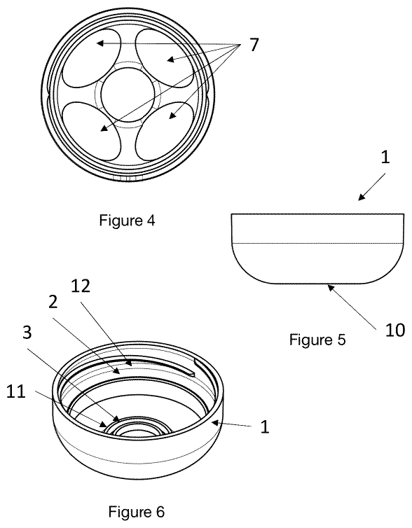

The seals 2 and 3 are fixed inside of the lid 1 so that they can be compressed when screwed on to the main body 4, therefore making the product watertight. As is shown in FIG. 5, the bottom 10 of lid 1 is flat so that the case can rest on any horizontal surface without falling over.

FIGS. 3, 4, and 7 show funnel end 9 has several points where it is provided with raised internal formations 7 inside the main body 4 so that when the opening of the menstrual cup 13 sits on top of those points 7 there is room for liquid to flow underneath to drain out from the funnel end 9 of the main body 4. The formations 7 can also be recesses.

The practice for using the case described above to clean a menstrual cup would be for the user to hold the stem end of the menstrual cup 14 and insert the open end of the menstrual cup 13 into the opening end 8 of the main body 4. The fully assembled unit seen in FIG. 2 is water-tight due to seals 2 and 3 and would be filled with liquid by the user prior to use. The lid 1 on the opening end 8 of the main body 4 indicated by the large opening of the notch 16 would be screwed off first, then a menstrual cup is placed open side first 13 into the opening end 8 of the main body 4. The lid 1 is then screwed back on the main body 4. The user then shakes the product to clean the menstrual cup. Then the lid 1 of the funnel end 9 of the main body 4 indicated by the point the notch 15 is screwed off and the liquid is then drained out through the funnel end 9 of the main body 4. The divots or formations 7 in the funnel end 9 of the main body 4 ensure that all the liquid can be drained out and that nothing gets stopped by the menstrual cup. The currently removed lid 1 is then screwed back on the funnel end 9 of the main body 4, and lid 1 on the opening end 8 of the main body 4 is then screwed off so the user can remove the menstrual cup from the main body 4 via the stem end 14. The currently removed lid 1 is then screwed back on and the process is complete.

For sterilisation, starting with the fully assembled unit seen in FIG. 2, the lid 1 on the opening end 8 of the main body 4 indicated by the larger end of the notch 16 is removed, the menstrual cup is put open side first 13 into the main body 4, the main body 4 is then filled with water so that the cup is fully submerged. This is then placed in a microwave without putting lid 1 back on to allow the steam to escape. The unit is heated in the microwave on high power for approximately 3 minutes. After the unit has cooled sufficiently, the user can take the menstrual cup out, pour the water out, dry the unit and store safely.

Further changes can be made within the scope of the invention.

1. A container for a menstrual cup comprising an open end and a closed end, the container comprising:
a tubular body including:
a first end with an opening that is wide enough to admit the open end of a menstrual cup; and
a second end at which the tubular body tapers inwardly to define an inner surface for supporting the open end of a menstrual cup inserted into the body, wherein formations are defined in the inner surface such that when the open end of a menstrual cup is supported on the inner surface, a fluid communication path is provided between the inside of the tubular body around the outside of the menstrual cup and a space in front of the open end of the menstrual cup;
a first end cap that is removably attached to the first end of the tubular body in a fluid-tight manner such that when the first end cap is attached to the tubular body, the first end cap, tubular body, and tapering inner surface define a space within which a menstrual cup can be contained; and
a second end cap that is removably attached over the second end of the tubular body in a fluid-tight manner;
such that the tubular body with the first and second end caps attached in a fluid-tight manner define a fluid-tight container for a menstrual cup.

2. A container as claimed in claim 1, wherein the second end of the tubular body includes an opening that is narrower than the opening at the first end and is not wide enough to admit the open end of a menstrual cup.

3. A container as claimed in claim 2, wherein the second end cap includes a seal that seals around the opening in the second end of the tubular body in a fluid-tight manner when the second end cap is attached to the second end of the tubular body.

4. A container as claimed in claim 3, wherein the first and second end caps are substantially the same and each include an outer seal for sealing around an outer part of the tubular body, and an inner seal for sealing around the opening in the second end.

5. A container as claimed in claim 1, wherein the first and second end caps are each flat in a central area.

6. A container as claimed in claim 1, wherein the formations in the inner surface are raised portions or recessed portions spaced around the inner surface.

7. A container as claimed in claim 1, wherein the tubular body includes an indicator on its outer surface showing which end is the first end and which end is the second end.

8. A container as claimed in claim 1, further comprising an elastomeric sleeve around the tubular body.

9. A container as claimed in claim 8, wherein the elastomeric sleeve and the tubular body have inter-engaging notches.

10. A container as claimed in claim 9, wherein the tubular body has a first ridge around its outer surface with a notch formed in the first ridge; and the elastomeric sleeve has a corresponding notch-shaped cutout in its edge at one end.

11. A container as claimed in claim 10, wherein the first ridge is adjacent the first end of the tubular body.

12. A container as claimed in claim 11, wherein the tubular body has a second ridge around its outer surface spaced from the first ridge and adjacent the second end of the tubular body, wherein the elastomeric sleeve is located between the first and second ridges.

13. A container as claimed in claim 9, wherein the notch on the tubular body has a wide opening and a point, and wherein the wide part is closer to the first end of the tubular body and the point is closer to the second end of the tubular body.

14. A method of using a container as claimed in claim 1, comprising:
filling the container with a cleaning liquid;
removing the first end cap;
inserting a menstrual cup through the opening in the first end so that the open end is supported on the inner surface at the second end;
attaching the first end cap to the tubular body;
shaking the container to clean the menstrual cup with the cleaning liquid;
opening the first end cap;
removing the menstrual cup; and
reattaching the first end cap.

15. A method of using the container of claim 1, comprising:
removing the first end cap;
inserting a menstrual cup through the opening in the first end so that the open end is supported on the inner surface at the second end;
filling the container with a cleaning liquid;
heating the container and menstrual cup to a sterilising temperature for a period of time;
removing the menstrual cup; and
draining the cleaning liquid from the container.
